(19) United States
(12) Patent Application Publication
(10) Pub. No.: US 2025/0009939 A1
(43) Pub. Date: Jan. 9, 2025

(54) VASCULAR BONE ORGANOID AND ITS COMPRESSION-PERFUSION FABRICATOR SYSTEM

(71) Applicant: National Defense Medical Center, Taipei (TW)

(72) Inventors: Jia-Fwu SHYU, New Taipei (TW); Tzu-Hui CHU, Taipei (TW); YU-CHIH LO, New Taipei (TW); YI-TING LAI, Taipei (TW); YU-MIN TSAI, Tainan (TW); Yu-Cheng KANG, Kaohsiung (TW)

(21) Appl. No.: 18/761,801

(22) Filed: Jul. 2, 2024

(30) Foreign Application Priority Data
Jul. 7, 2023 (TW) 112125512

(51) Int. Cl. A61L 27/38; C12M 1/00; C12M 1/12; C12M 1/24; C12M 1/36; C12M 3/00; C12N 5/071

(52) U.S. Cl. CPC A61L 27/3895; A61L 27/3808; A61L 27/3821; A61L 27/3847; A61L 27/3891; C12M 21/08; C12M 23/08; C12M 23/48; C12M 25/14; C12M 29/10; C12M 41/48; C12N 5/0697; A61L 2430/02; C12N 2513/00; C12N 2533/30; C12N 2533/54

(57) ABSTRACT

The present invention discloses a vascular bone organoid and its compression-perfusion fabricator system. The compression-perfusion fabricator system includes a compression device, a perfusion chamber, a perfusion device, and a microcomputer driver. The compression device and the perfusion device are controlled and adjusted through the microcomputer driver to simultaneously provide dynamic mechanical compression and perfusion stimulation. The system simulates the dynamic microenvironment within the human body. The present invention further utilizes 3D bioprinting technology to manufacture a vascular bone organoid, which is cultured in the compression-perfusion fabricator system, to mimic the growth of bone and vascular endothelial cells within the normal dynamic physiological environment of the bone marrow cavity.

### CROSS-REFERENCE TO RELATED APPLICATIONS

This non-provisional application claims priority under 35 U.S.C. § 119 (a) on Patent Application No(s). 112125512 filed in Taiwan on Jul. 7, 2023, the entire contents of which are hereby incorporated by reference.

### BACKGROUND OF THE INVENTION

### 1. Field of the Invention

The present invention relates to cell culture technology, specifically to a vascular bone organoid and its compression-perfusion Fabricator system.

### 2. Description of the Prior Art

The concept and technology of organoids have been gradually developing in recent years. Organoids are primarily generated using stem cells to create 3D structures that mimic the functions of human organs and tissues. Compared with traditional 2D cell cultures or animal models, organoids represent the physiological model of human organs more accurately. Organoids can serve as models for studying organ development, disease mechanisms, drug screening, and personalized medicine. In the fields of drug development and pathology research, organoids provide a powerful tool for investigating the molecular and cellular mechanisms of diseases and developing new treatments and therapies. Moreover, organoids offer realistic models for drug efficacy and toxicity testing and can be used in personalized medicine allowing clinicians to test potential treatments on the patient's cells before administering them. This technology enables faster and more accurate diagnoses, as well as the development of more effective treatments with fewer side effects.

Bone organoids with a similar structure to real bone tissue are fabricated through tissue engineering and stem cell technology. Bone organoids can simulate the structure, function, and physiological characteristics of bones, aiding in studying skeletal development, disease progression, and related treatment methods. For example, osteoporosis and its associated fractures have always been a significant global health issue, yet there are currently no ideal treatments for osteoporosis, with most developed drugs having cardiovascular side effects. One major reason for the above problem is the lack of a good research model. Most of the existing drug development models overly focus on the effects on bone tissue while neglecting the effects on other tissues. Osteogenesis and angiogenesis are inseparable, and the development of osteoporosis drugs must consider both the impact on bone tissue and vasculature. Bone organoids, created using tissue engineering technology and biological principles, represent a new in vitro concept capable of simulating complex in vivo organ functions and have the potential to become important models for pathology or drug development in the future.

However, most of the relevant researches are currently difficult to make progress due to bone organoids requiring mineralization to increase the hardness and strength. Additionally, the complex distribution of vascular networks within the bone marrow cavity poses a significant obstacle to integrating bone cells with vascular endothelial cells to mimic the actual organ structure within the human body remains an intractable problem in this field.

Furthermore, in the physiological environment within the body, actual bone organs will be affected by various solid and fluid physical forces such as pressure, tension, torsion, and shear stress. Multiple studies have indicated that mechanical stimuli can alter the shape of bone organs and enhance their hardness and strength. However, the mechanisms by which physical forces are translated into molecular and biological signals remain unclear. Therefore, to develop effective and physiologically relevant models for pathological or drug development studies, overcoming the challenge of accurately simulating the complex bone tissue environment ex vivo, including mechanical forces, fluid shear stress, and tissue architecture, is urgently needed for the development of organoids.

### SUMMARY OF THE INVENTION

The present invention provides a compression-perfusion fabricator system, comprising:
a compression device comprising a cell growth area, a first electric gripper jaw, a second electric gripper jaw, and a micromotor, wherein the cell growth area is positioned between the first and second electric gripper jaws and the micromotor is electrically connected to the first and second electric gripper jaws;
a perfusion chamber for loading cell culture medium, wherein the cell growth area and the first and second electric gripper jaws are arranged in the perfusion chamber;
a perfusion device comprising a culture medium bottle, a culture medium recycling bottle, and a peristaltic pump, wherein the peristaltic pump is connected to the perfusion chamber, the culture medium bottle, and the culture medium recycling bottle through tubes; and
a microcomputer driver comprising a control panel, wherein the microcomputer driver is electrically connected to the peristaltic pump and the micromotor;
wherein the compression device and the perfusion device are controlled and adjusted through the microcomputer driver to simulate the dynamic microenvironment in the human body.

In some embodiments, the system further comprises at least one support bracket for fixing the compression device, the perfusion chamber, and the perfusion device.

In some embodiments, the microcomputer driver further comprises a flow sensor.

In some embodiments, the system is used for culturing and differentiating at least one stem cell, wherein the stem cell is selected from the group consisting of osteoprogenitor cells (OPCs), human umbilical vein endothelial cells (HUVEC), mesenchymal stem cells (MSC), and hematopoietic stem cells.

In some embodiments, the perfusion device applies pressure through the peristaltic pump to transport fresh culture medium from the culture medium bottle into the perfusion chamber via tubing, and to transport the culture medium from the perfusion chamber into the culture medium recycling bottle via tubing.

In some embodiments, the microcomputer driver controlled the peristaltic pump by adjusting control parameters displayed on the control panel.

In some embodiments, the control parameters comprise liquid flow rate, liquid flow volume, and perfusion time.

In some embodiments, the compression device shortens the distance between the first electric gripper jaw and the second electric gripper jaw through the micromotor to generate pressing force on the cell growth area.

In some embodiments, the microcomputer driver controlled the micromotor by adjusting control parameters displayed on the control panel.

In some embodiments, the control parameters comprise the position of the first electric gripper jaw, the position of the second electric gripper jaw, the compression time of the first and second electric gripper jaws, the compression rate of the first and second electric gripper jaws, the amount of the compressive force, the duration of the compressive force, the frequency of the compressive force, and the amount of deformation.

In some embodiments, the compression device secures an organoid in the cell growth area with the first and second electric gripper jaws and immerses the organoid in the perfusion chamber.

In some embodiments, the organoid is a vascular bone organoid, comprising:
a first osteogenic mesh scaffold seeded with osteoprogenitor cells (OPCs);
a second osteogenic mesh scaffold seeded with osteoprogenitor cells (OPCs); and
and placed between the first and second osteogenic mesh scaffolds.

In some embodiments, the compression-perfusion fabricator system is used for culturing an organoid.

In one aspect, the present invention provides a vascular bone organoid, comprising:
a first osteogenic mesh scaffold made of a first biocompatible material, wherein the first osteogenic mesh scaffold is seeded with osteoprogenitor cells (OPCs);
a second osteogenic mesh scaffold made of a second biocompatible material, wherein the second osteogenic mesh scaffold is seeded with osteoprogenitor cells (OPCs); and
a vascular mesh scaffold placed between the first and second osteogenic mesh scaffolds, wherein the vascular mesh scaffold is made of a third biocompatible material and seeded with human umbilical vein endothelial cells (HUVEC).

In some embodiments, the first osteogenic mesh scaffold, the second osteogenic mesh scaffold, and the vascular mesh scaffold are made by 3D printing.

In some embodiments, the first, second, and third biocompatible materials are independently selected from the group consisting of polycaprolactone, gelatin methacryloyl (GelMa), Pluronic F127, chitosan, collagen, and alginate.

In some embodiments, the vascular bone organoid is cultured through the compression-perfusion fabricator system as described in the present invention, wherein the vascular bone organoid is cultured in the cell growth area.

In another aspect, the present invention provides a method for culturing organoids, comprising:
providing a compression-perfusion fabricator system as described in the present invention;
culturing at least one stem cell on the cell growth area; and
utilizing the peristaltic pump for fluid perfusion and employing the micromotor for compressive stimulation, to simulate the dynamic microenvironment of cell culture in the human body.

In some embodiments, the stem cell is selected from the group consisting of osteoprogenitor cells (OPCs), human umbilical vein endothelial cells (HUVECs), mesenchymal stem cells (MSCs), and hematopoietic stem cells.

In some embodiments, the method is used for culturing the vascular bone organoid as described in the present invention, wherein the vascular bone organoid is cultured in the cell growth area.

Compared with traditional 2D cell culture, the vascular bone organoid disclosed in the present invention combines 3D bioprinting technology and stem cell generation to simulate the 3D structure and functions of human bone organs, which more accurately reflect the actual physiological conditions of human organs.

Furthermore, compared with traditional static culture, the compression-perfusion fabricator system disclosed in the present invention allows for precise simulation of the physiological environment of human bone tissue, including mechanical forces and fluid shear stress. This is achieved through a microcomputer driver that adjusts parameters such as compression force, compression duration, compression frequency, perfusion flow rate, and perfusion velocity. By utilizing the compression-perfusion fabricator system, researchers can conduct more accurate and effective studies on bone tissue disease models, drug screening, and molecular mechanisms.

The present invention can further utilize a patient's bone marrow cells and cooperate with the scaffold materials, to fabricate vascular bone organoids through 3D bioprinting. Under the stimulation of mechanical compression and perfusion fluid systems, this approach can rapidly promote vascularization within bone tissue and enhance extracellular matrix mineralization (increasing bone tissue hardness). Additionally, the compression-perfusion fabricator system serves as an in vitro research instrument that simulates the normal living environment of bone cells in vivo. This is advantageous for solving the problem of detailed mechanisms by which mechanical and fluid forces stimulate bone and vascular formation, thereby contributing to the development of more effective and safer treatments for osteoporosis and related fractures.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows the schematic diagram of the compression-perfusion fabricator system.

FIGS. 2A and 2B show the schematic front view diagrams illustrating the compression device before (2A) and after (2B) generating compressive force.

FIG. 3 shows the schematic front view diagram of the compression device equipped with a vascular bone organoid.

FIG. 4 shows the schematic diagram of the structure of the vascular bone organoid.

FIG. 5 shows the survival and proliferating cell numbers of HUVACs and OPCs in the vascular bone organoid.

FIGS. 6A and 6B show the micrographs of human umbilical vein endothelial cells (HUVECs) growing on the surface of a GelMA vascular mesh scaffold. FIG. 6A shows HUVECs labeled by immunofluorescence staining (blue); FIG. 6B shows the result of DAPI nuclear staining (blue). The scale bar is 30 μm.

FIG. 7 shows the survival rate of human umbilical vein endothelial cells (HUVECs) in the vascular bone organoid after compressive stimulation.

FIG. 8 shows the confocal micrograph of HUVECs with a functional marker (CD31, green) on a GelMA vascular mesh scaffold. The cell nuclei were visualized by DAPI staining (blue). * indicates the location of the scaffold pore. The scale bar is 100 μm.

FIGS. 9A, 9B, 9C, and 9D show the micrographs of osteoblasts growing on the surface of a polycaprolactone (PCL) osteogenic mesh scaffold. FIG. 9A shows the cytoskeleton of osteoblasts labeled by immunofluorescence staining (red); FIG. 9B shows the results of DAPI nuclear staining (blue); FIG. 9C shows the pore of the PCL osteogenic mesh scaffold observed under phase-contrast microscopy; FIG. 9D shows the expression of alkaline phosphatase by osteoblasts on the PCL osteogenic mesh scaffold observed under optical microscopy. * indicates the location of the scaffold pore. The scale bar is 30 μm.

FIGS. 10A, 10B, and 10C show the protein expression of HUVECs after compressive stimulation using the compression-perfusion fabricator system described in the present invention. FIG. 10A shows the expression of PIEZO1. FIG. 10B shows the expression of endomucin (EMCN). FIG. 10C shows the phosphorylation results of the YAP protein.

FIGS. 11A and 11B show the RNA expression of HUVECs between mono-culture and co-culture after compressive stimulation using the compression-perfusion fabricator system described in the present invention. FIG. 11A shows the expression of PIEZO1. FIG. 11B shows the expression of endomucin (EMCN).

### DETAILED DESCRIPTION OF THE PREFERRED EMBODIMENT

In view of the problems and shortcomings of traditional techniques, the present invention utilizes 3D bioprinting and cell culture technologies to fabricate a vascular bone organoid. The vascular bone orgnoid combined with the compression-perfusion fabricator system, which provides mechanical stimulation through compression and perfusion forces simulates the effects of the real in vivo environment, offering an effective model for drug screening and pathological research.

It should be understood that both the foregoing general description and the following detailed description are exemplary and explanatory but are not restrictive of the invention as claimed. Certain details of one or more embodiments of the invention are set forth in the description below. Other features or advantages of the present invention will be apparent from the non-exhaustive list of representative examples that follows, and also from the appending claims.

Unless otherwise defined herein, scientific and technical terms used in connection with the present disclosure shall have the meanings that are commonly understood by those of ordinary skill in the art.

As used herein, the singular form “a”, “an”, and “the” includes plural references unless indicated otherwise. For example, “an” excipient includes one or more excipients. Unless explicitly stated otherwise in the context, the terms “or” and “and/or” can be used interchangeably.

As used interchangeably herein, “around”, “about” and “approximately” shall generally mean plus or minus 10% of the numerical value of the number with which it is being used. Therefore, about 1% means in the range of 0.9% to 1.1%. Numerical quantities given herein are approximate, meaning that the term “around”, “about” or “approximately” can be inferred if not expressly stated.

The phrase “comprising” as used herein is open-ended, indicating that such embodiments may include additional elements. In contrast, the phrase “consisting of” is closed, indicating that such embodiments do not include additional elements (except for trace impurities). The phrase “consisting essentially of” is partially closed, indicating that such embodiments may further comprise elements that do not materially change the basic characteristics of such embodiments.

Unless otherwise required by context, singular terms shall include pluralities and plural terms shall include the singular. The methods and techniques of the present disclosure are generally performed according to conventional methods well-known in the art. Generally, nomenclatures used in connection with, and techniques of biochemistry, enzymology, molecular and cellular biology, microbiology, medicine, pharmacy, genetics, and protein and nucleic acid chemistry, hybridization, and 3D printing described herein are those well-known and commonly used in the art. The methods and techniques of the present disclosure are generally performed according to conventional methods well known in the art and as described in various general and more specific references that are cited and discussed throughout the present specification unless otherwise indicated.

To facilitate the understanding of the technical features, content, advantages, and efficacy of the present invention, the invention is described in detail below with reference to the accompanying drawings and in the form of embodiments. The purpose of the drawings is only for illustration and auxiliary description, and may not necessarily represent the actual proportions and precise configurations of the invention as implemented. Therefore, the proportions and spatial relationships shown in the drawings should not be interpreted as limiting the scope of the present invention in actual implementation.

The present invention provides a compression-perfusion fabricator system, comprising:
a compression device comprising a cell growth area, a first electric gripper jaw, a second electric gripper jaw, and a micromotor, wherein the cell growth area is positioned between the first and second electric gripper jaws and the micromotor is electrically connected to the first and second electric gripper jaws;
a perfusion chamber for loading cell culture medium, wherein the cell growth area and the first and second electric gripper jaws are arranged in the perfusion chamber;
a perfusion device comprising a culture medium bottle, a culture medium recycling bottle, and a peristaltic pump, wherein the peristaltic pump is connected to the perfusion chamber, the culture medium bottle, and the culture medium recycling bottle through tubes; and
a microcomputer driver comprising a control panel, wherein the microcomputer driver is electrically connected to the peristaltic pump and the micromotor;
wherein the compression device and the perfusion device are controlled and adjusted through the microcomputer driver to simulate the dynamic microenvironment in the human body.

In some embodiments, the system further comprises at least one support bracket for fixing the compression device, the perfusion chamber, and the perfusion device.

In some embodiments, the microcomputer driver further comprises a flow sensor.

In some embodiments, the system is used for culturing and differentiating at least one stem cell, wherein the stem cell is selected from the group consisting of osteoprogenitor cells (OPCs), human umbilical vein endothelial cells (HUVEC), mesenchymal stem cells (MSC), and hematopoietic stem cells.

In some embodiments, the perfusion device applies pressure through the peristaltic pump to transport fresh culture medium from the culture medium bottle into the perfusion chamber via tubing, and to transport the culture medium from the perfusion chamber into the culture medium recycling bottle via tubing.

In some embodiments, the microcomputer driver controlled the peristaltic pump by adjusting control parameters displayed on the control panel.

In some embodiments, the control parameters comprise liquid flow rate, liquid flow volume, and perfusion time.

In some embodiments, the compression device shortens the distance between the first electric gripper jaw and the second electric gripper jaw through the micromotor to generate pressing force on the cell growth area.

In some embodiments, the microcomputer driver controlled the micromotor by adjusting control parameters displayed on the control panel.

In some embodiments, the control parameters comprise the position of the first electric gripper jaw, the position of the second electric gripper jaw, the compression time of the first and second electric gripper jaws, the compression rate of the first and second electric gripper jaws, the amount of the compressive force, the duration of the compressive force, the frequency of the compressive force, and the amount of deformation.

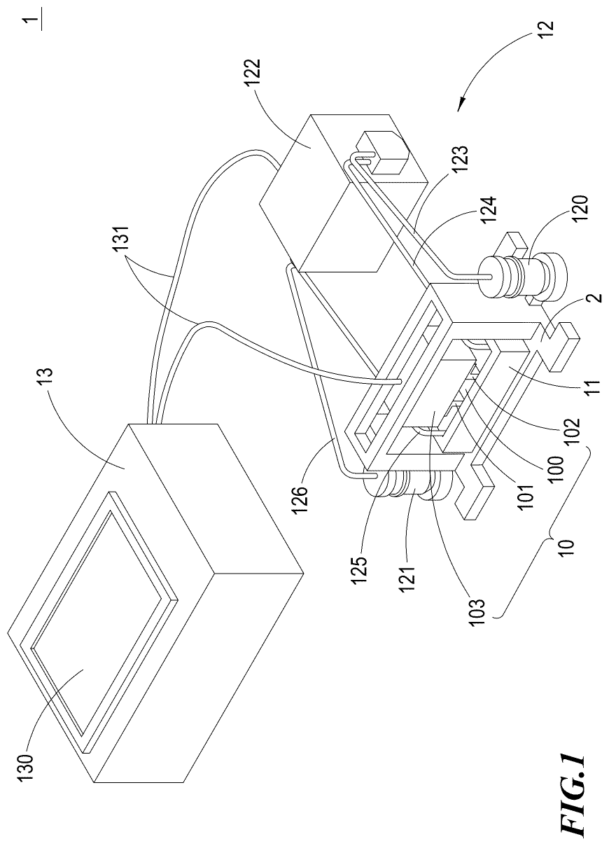

Please refer to FIG. 1, which is a schematic diagram of the compression-perfusion fabricator system 1 described in the present invention. The micromotor 103 in the compression device 10 is held by the support bracket 2, allowing the cell growth area 100, the first electric gripper 101, and the second electric gripper 102 to be immersed in the perfusion chamber 11. Additionally, the micromotor 103 is electrically connected to the microcomputer driver 13 via a wire 131. On the other hand, in the perfusion device 12, the culture medium bottle 120 and the culture medium recycling bottle 121 are fixed by the support bracket 2. The peristaltic pump 122 suctions out the culture medium from the culture medium bottle 120 through a first tube 123 and then pumps it into the perfusion chamber 11 through a second tube 124. The peristaltic pump 122 also suctions out the culture medium from the perfusion chamber 11 through a third tube 125 and then pumps it into the culture medium recycling bottle 121 through a fourth tube 126. Additionally, the peristaltic pump 122 is electrically connected to the microcomputer driver 13 via a wire 131.

The microcomputer driver 13 is operated through the control panel 130 to control the parameters of the micromotor 103 and the peristaltic pump 122, thereby regulating the compression mechanics and perfusion mechanics stimulation in the compression-perfusion fabricator system of the present invention, and simulating the real dynamics physiological environment in the body. The parameters that the microcomputer driver 13 can control in the micromotor 103 include but are not limited to the position of the first electric gripper jaw, the position of the second electric gripper jaw, the compression time of the first and second electric gripper jaws, the compression rate of the first and second electric gripper jaws, the amount of the compressive force, the duration of the compressive force, the frequency of the compressive force, and the amount of deformation. The parameters that the microcomputer driver 13 can control in the peristaltic pump 122 include but are not limited to liquid flow rate, liquid flow volume, and perfusion time. In some embodiments, the microcomputer driver 13 further comprises a flow sensor to detect parameters such as the flow rate and flow rate of the liquid.

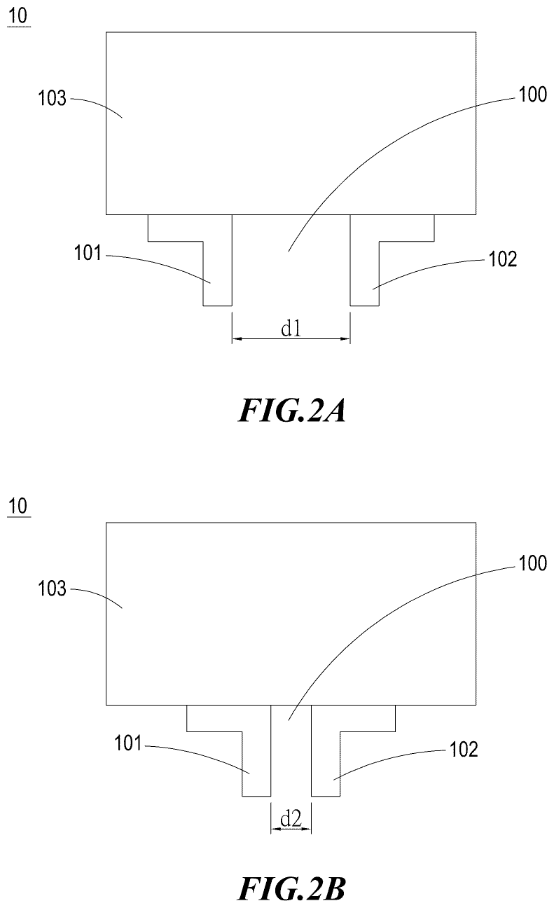

Please refer to FIGS. 2A and 2B, which are the schematic front view diagrams illustrating the compression device 10 of the present invention before and after generating compressive force, respectively. The area between the first electric gripper jaw 101 and the second electric gripper jaw 102 is the cell growth area 100. As shown in FIG. 2A, the distance between the first electric gripper jaw 101 and the second electric gripper jaw 102 is d1, which depends on the positions of the first electric gripper jaw 101 and the second electric gripper jaw 102. These positions are regulated by the micromotor 103. As shown in FIG. 2B, when the micromotor 103 adjusts the positions of the first electric gripper jaw 101 and the second electric gripper jaw 102, causing them to move closer together, the distance changes to d2 (d1>d2), resulting in a compressive force stimulus on the cell growth area 100. By operating the microcomputer driver, the parameters such as the position of the first electric gripper jaw, the position of the second electric gripper jaw, the contraction duration of the first and second electric gripper jaws, and the contraction rate of the first and second electric grippers can be controlled, thereby generating varying magnitudes of compressive force, durations of compressive force, and frequencies of compressive force.

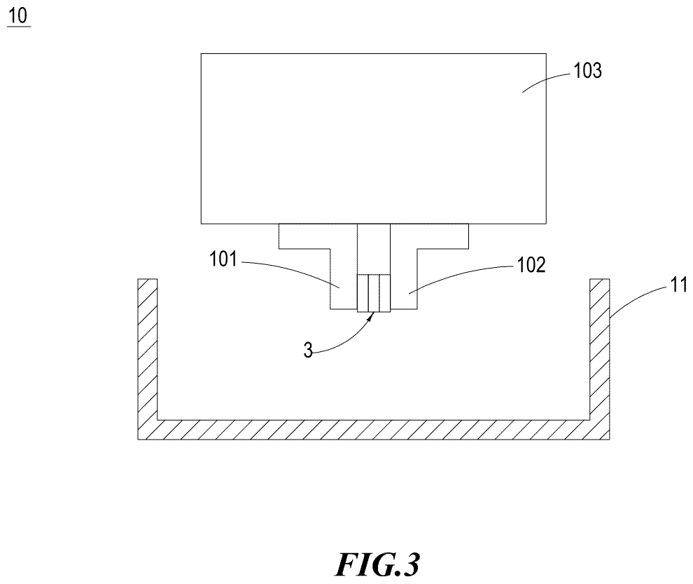

Please refer to FIG. 3, which is the schematic front view diagram of the compression device 10 of the present invention equipped with a vascular bone organoid. As shown in FIG. 3, the vascular bone organoid 3 is fixed by the first electric gripper jaw 101 and the second electric gripper jaw 102 and immersed in the perfusion chamber 11. At the same time, the positions of the first electric gripper jaw 101 and the second electric gripper jaw 102 are controlled by the micromotor 103 to generate a compressive force on the vascular bone organoid 3. By operating the microcomputer driver to control the micromotor 103, the parameters such as the amount, duration, and frequency of the compressive force can be controlled, thereby simulating the compressive force of the dynamic physiological environment in which bone cells are subjected to in vivo.

In some embodiments, the compression device secures an organoid in the cell growth area with the first and second electric gripper jaws and immerses the organoid in the perfusion chamber.

In some embodiments, the organoid is a vascular bone organoid, comprising:
a first osteogenic mesh scaffold seeded with osteoprogenitor cells (OPCs);
a second osteogenic mesh scaffold seeded with osteoprogenitor cells (OPCs); and and placed between the first and second osteogenic mesh scaffolds.

In some embodiments, the compression-perfusion fabricator system is used for culturing an organoid.

In one aspect, the present invention provides a vascular bone organoid, comprising:
a first osteogenic mesh scaffold made of a first biocompatible material, wherein the first osteogenic mesh scaffold is seeded with osteoprogenitor cells (OPCs);
a second osteogenic mesh scaffold made of a second biocompatible material, wherein the second osteogenic mesh scaffold is seeded with osteoprogenitor cells (OPCs); and
a vascular mesh scaffold placed between the first and second osteogenic mesh scaffolds, wherein the vascular mesh scaffold is made of a third biocompatible material and seeded with human umbilical vein endothelial cells (HUVEC).

In some embodiments, the first osteogenic mesh scaffold, the second osteogenic mesh scaffold, and the vascular mesh scaffold are made by 3D printing.

In some embodiments, the first, second, and third biocompatible materials are independently selected from the group consisting of polycaprolactone, gelatin methacryloyl (GelMa), Pluronic F127, chitosan, collagen, and alginate.

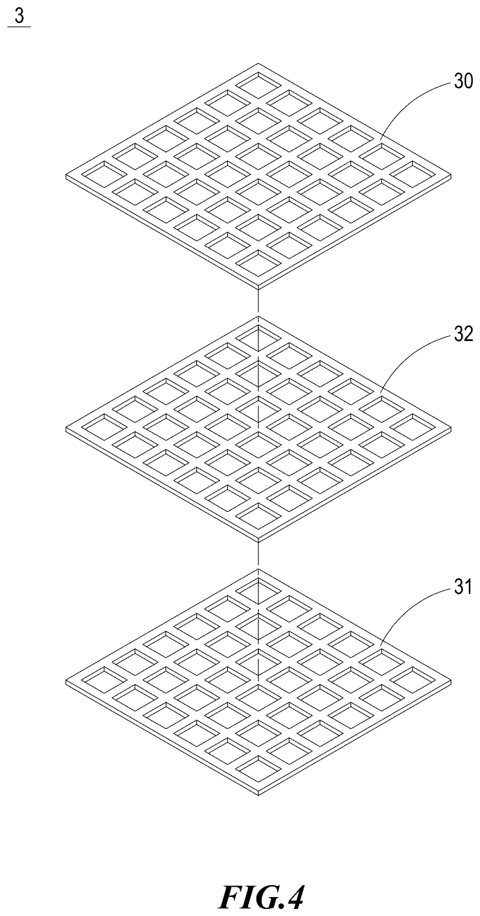

Please refer to FIG. 4, which is the schematic diagram of the structure of the vascular bone organoid 3 of the present invention. A vascular mesh scaffold 32 is provided between the first osteogenic mesh scaffold 30 and the second osteogenic mesh scaffold 31. The first osteogenic mesh scaffold 30, the second osteogenic mesh scaffold 31, and the vascular mesh scaffold 32 are made by printing biocompatible materials into a hollow cubic mesh structure through 3D bioprinting technology to serve as the bone scaffold for cell culture. Each hollow cubic mesh structure is approximately 1 cm² and has a thickness of approximately 0.2 cm.

In some embodiments, the vascular bone organoid is cultured through the compression-perfusion fabricator system as described in the present invention, wherein the vascular bone organoid is cultured in the cell growth area.

In another aspect, the present invention provides a method for culturing organoids, comprising:
providing a compression-perfusion fabricator system as described in the present invention;
culturing at least one stem cell on the cell growth area; and
utilizing the peristaltic pump for fluid perfusion and employing the micromotor for compressive stimulation, to simulate the dynamic microenvironment of cell culture in the human body.

In some embodiments, the stem cell is selected from the group consisting of osteoprogenitor cells (OPCs), human umbilical vein endothelial cells (HUVECs), mesenchymal stem cells (MSCs), and hematopoietic stem cells.

In some embodiments, the method for culturing organoids is used for culturing the vascular bone organoid as described in the present invention, wherein the vascular bone organoid is cultured in the cell growth area.

As used herein, the term “dynamic microenvironment” refers to the constantly changing and interacting local environments that surround cells and tissues. They are influenced by various factors such as biochemical signals, physical forces, cellular interactions, and the presence of extracellular matrix components.

The present invention is further illustrated by the following examples, which are provided for the purpose of demonstration rather than limitation. Those of skill in the art should, in light of the present disclosure, appreciate that many changes can be made in the specific embodiments which are disclosed and still obtain a like or similar result without departing from the spirit and scope of the invention.

### EXAMPLE 1 Process for Producing Vascular Bone Organoid

In this example, the producing process of the vascular bone organoid is divided into preparing the osteogenic mesh scaffold and prepareing the vascular mesh scaffold.

### 1.1 the Preparation of the Osteogenic Mesh Scaffold

In this embodiment, the first osteogenic mesh scaffold and the second osteogenic mesh scaffold of the vascular bone organoid are made through 3D scaffold printing technology, wherein the osteoprogenitor cells (OPCs) are cultured on the scaffold through 3D cell culture technology. The specific steps comprise: Designing the scaffold model using 3D computer-aided design software (Tinkercad) for subsequent 3D printing.

Filling the hot-melt extruder with polycaprolactone (PCL) pellets and installing the extruder onto the BioX bioprinter (Cellink, Sweden).

Using a 27G stainless steel nozzle for printing with the following parameters: 25% density, a temperature of 180° C., a pressure of 200 kPa, and a speed of 1.2 mm/s. After printing, allow the PCL to cool and solidify, changing from transparent to white, then remove it from the culture dish.

Sterilizing the printed PCL scaffold by soaking it in 75% ethanol for 10 minutes. Then, soak the PCL scaffold in a 1:1000 Poly-L-Lysine solution and place it in an incubator at 37° C. and 5% CO₂ for one hour to enhance cell adhesion. Wash the scaffold three times with PBS.

Seeding 50 μL of cell suspension containing 2×10⁵ OPCs onto the PCL scaffold. After one hour, adding osteoblast medium (ObM) and incubating overnight. Sandwiching a GelMA scaffold without cells between two PCL scaffolds containing OPCs, and adding 1 mL of ObM for incubating. Change the medium every three days.

### 1.2 the Preparation of the Vascular Mesh Scaffold

In this embodiment, the vascular mesh scaffold of vascular bone organoid is fabricated using 3D scaffold printing technology. Subsequently, human umbilical vein endothelial cells (HUVECs) were seeded and cultured using 3D cell culture technology. The specific steps comprise:

Designing the scaffold model using 3D computer-aided design software (Tinkercad) for subsequent 3D printing.

Preparing gelatin methacryloyl (GelMA) bio-ink at a concentration of 3 mL containing 1×10⁷ HUVECs. Mix the GelMA and HUVEC cell suspension in a 10:1 ratio using a syringe and a specialized adapter to ensure uniform mixing. Injecting the mixture into a sterilized specialized ink cartridge to complete the preparation of the bio-ink. Installing the temperature-controlled nozzle to the BioX bioprinter and loading the ink cartridge containing the GelMA bio-ink described above. Using a 27G plastic needle for printing with the following parameters: 40% density, temperature range of 20 to 25° C., pressure range of 85 to 110 kPa, and a speed of 2 mm/s. After printing, expose to 405 nm UV light for 30 seconds to crosslink and solidify the GelMA scaffold.

After UV cross-linking, the GelMA scaffold can be easily removed from the culture dish by adding phosphate buffered saline (PBS). Transferring the scaffold to a 24-well plate and adding endothelial cell medium (ECM) for 37° C. incubating with 5% CO₂. The next day, sandwiched a GelMA scaffold containing HUVECs between two sterilized PCL cell-free scaffolds and added 1 mL of ECM for incubating. Change the medium every three days.

### 1.3 the Preparation of the Vascular Bone Organoid (Co-Culture)

Following the aforementioned process, taking one vascular mesh scaffold (GelMA scaffold containing HUVECs) and sandwiching it between two osteogenic mesh scaffolds (PCL scaffolds containing OPCs), forming a structure as shown in FIG. 4, which is the vascular bone organoid described in the present invention. Culturing the combined structure to form the vascular bone organoid described in the present invention. Using the compression-perfusion fabricator system described in the present invention to culture the vascular bone organoid. Adding a mixture of endothelial cell medium (ECM) and osteoblast medium (ObM) to the perfusion chamber. Placing the setup in an incubator at 37° C. with 5% CO₂, and changing the medium every three days.

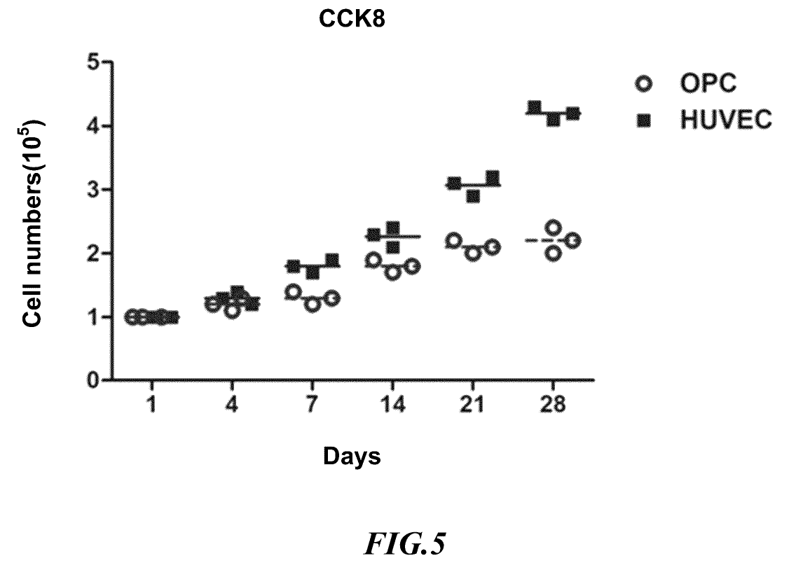

In this embodiment, cell numbers of HUVECs (on GelMA scaffold) and OPCs (on PCL scaffold) growing on the vascular bone organoid are counted through Cell Counting Kit-8 (CCK8, Dojindo, Japan, catalog number No. CK04-11) on day 1, 4, 7, 14, 21 and 28. Each sample is performed in triplicate (n=3). Please refer to FIG. 5, the cell numbers of HUVECs and OPCs gradually increase as the days of culture increase, indicating that cells can normally grow on the vascular bone organoid in the present invention.

### EXAMPLE 2 HUVECs of the Vascular Bone Organoid

In this example, human umbilical vein endothelial cells (HUVECs) were seeded onto the gelatin methacryloyl (GelMA) vascular mesh scaffold by the process described in Example 1. The growth of the HUVECs on the vascular mesh scaffold was observed. The vascular bone organoid described in the present invention is cultured using the compression-perfusion fabricator system of the present invention to apply compressive force and perfusion stimulation, thereby simulating the dynamic microenvironment in vivo. The cell viability of the HUVECs was observed after receiving the stimulation.

2.1 The cell growth of HUVECs

### Materials and Methods

In this embodiment, Human umbilical vein endothelial cells (HUVECs) were seeded onto the Gelatin Methacryloyl (GelMA) vascular mesh scaffold as described in Example 1.2 and cultured for 21 days. Immunofluorescence (IF) staining using a CD31 antibody was performed to label the HUVECs within the GelMA vascular mesh scaffold, combined with DAPI nuclear staining to observe cell growth. The specific experimental steps comprise:

Removing the culture medium from the GelMA vascular mesh scaffold and wash with PBS. Adding 4% paraformaldehyde and shaking for 40 minutes to immobilize the cells in the scaffold. Wash three times with PBS and shake for 10 minutes each time to remove the paraformaldehyde.

Covering the scaffold with primary antibody (Mouse/rat CD31/PECAM-1 antibody, Bio Basic, Canada, Catalog No. PB0684), diluted in blocking buffer at a ratio of 1:200, and incubate at 4° C. for three days.

Wash three times with PBS and shake for 10 minutes each time. Adding the secondary antibody (Donkey anti-goat IgG FITC, Jackson, USA, Catalog No. 705-095-003), diluted in blocking buffer at a ratio of 1:200. Covering with aluminum foil paper to protect from light, and incubating at room temperature for three days. Wash three times with PBS and shake for 10 minutes each time.

Staining the cell nuclei with DAPI (BioLegend, USA, Catalog No. 422801), diluted 1:10000 in PBS, covering the scaffold and shaking for 5 minutes. Subsequently, wash three times with PBS and shake for 5 minutes each time. Transfering the scaffold to a glass-bottom culture dish and adding fluorescent mounting medium to observe the results.

### Results

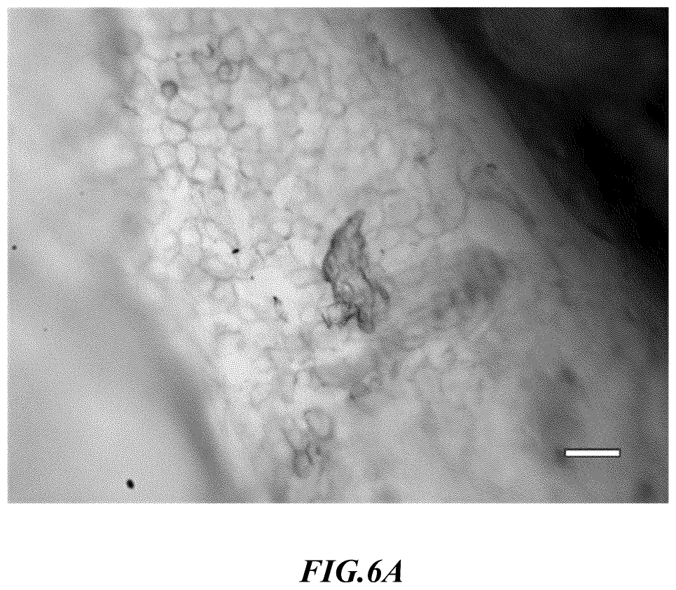

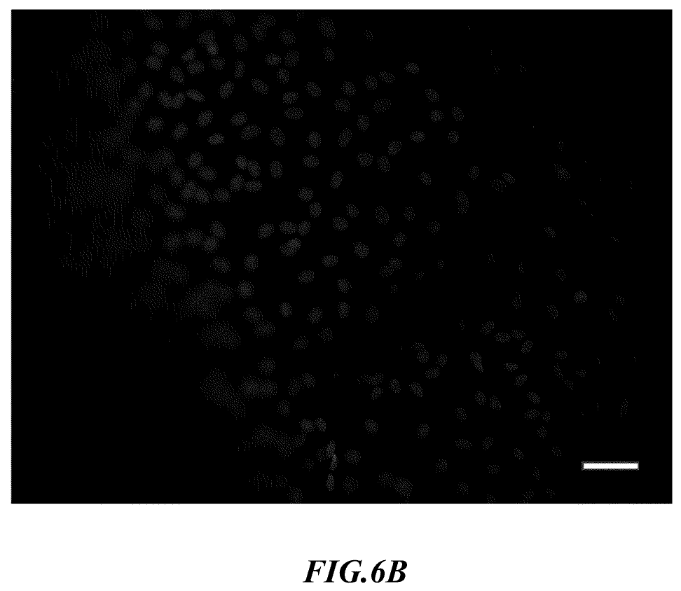

Please refer to FIGS. 6A and 6B, which are the micrographs of human umbilical vein endothelial cells (HUVECs) growing on the surface of a gelatin methacryloyl (GelMA) vascular mesh scaffold. The scale bar in the figures represents 50 μm. In FIG. 6A, the blue regions indicate HUVECs labeled by immunofluorescence staining, while in FIG. 6B, the blue regions result from DAPI nuclear staining. As shown in FIGS. 6A and 6B, HUVECs are able to survive and grow well in the GelMA vascular mesh scaffold described in the present invention.

2.2 The survival rate of HUVECs

### Materials and Methods

In this embodiment, the vascular bone organoid was cultured using the compression-perfusion fabricator system for 2 days. Subsequently, compressive force stimulation was applied, with daily stimulation for 2 hours over periods of 2, 5, and 7 days. Cell number was assessed using the Cell Counting Kit-8 (CCK-8, Dojindo, Japan, Catalog No. CK04-11) to observe the survival rate of HUVECs following the application of compressive force via the compression-perfusion fabricator system. The specific experimental steps comprise:

Adding culture medium mixed with ECM and ObM to the perfusion chamber of the compression-perfusion fabricator system described in the present invention. Adjusting the compression parameters to a deformation amount of 10%, a voltage of 10 V (frequency 0.2 Hz) to provide the compressive stimulation, and placing the compression-perfusion fabricator system in a 37° C. incubator with 5% CO₂.

Before cell counting, prepare a standard concentration curve. Prepare six 5 ml centrifuge tubes, adding 4 ml of medium to the first tube and 2 ml of medium to each of the remaining tubes. Adding 2×10⁶ cells to the first tube, mix well, then transfer 2 ml of the cell suspension to the second tube. Repeat the process sequentially up to the fifth tube. The sixth tube does not receive any cell suspension. The dilution results in standard samples with cell concentrations of 2×10⁶ cells/4 ml, 1×10⁶ cells/4 ml, 5×10⁵ cells/4 ml, 2.5×10⁵ cells/4 ml, 1.25×10⁵ cells/4 ml, and 0 cells/4 ml.

Loading the standard samples into a 24-well plate with duplicates of 1 ml per well, resulting in cell concentrations of 5×10⁵ cells/ml, 2.5×10⁵ cells/ml, 1.25×10⁵ cells/ml, 6.25×10⁴ cells/ml, 3.125×10⁴ cells/ml, and 0 cells/ml. Incubate the plate at 37° C. with 5% CO₂ for one day to allow cells to adhere to the wells, forming the standard concentration curve.

After culturing the vascular bone organoid, remove the medium from the scaffold, wash it with PBS, and transfer the scaffold to a 24-well plate. Mix 500 μL of medium with 50 μL of CCK-8 reagent (medium to CCK-8 reagent ratio of 10:1). Cover the scaffold with the CCK-8 mixture and incubate at 37° C. with 5% CO₂ for three hours.

Loading 100 μL of the well-mixed CCK-8 solution incubated for 3 hours into each well of a 96-well plate, with three replicates for each concentration. Measure the absorbance at 450 nm.

Based on the linear regression analysis of the absorbance results against the standard concentration curve, the number of cells on or within the scaffold can be extrapolated through the absorbance result of the sample.

### Results

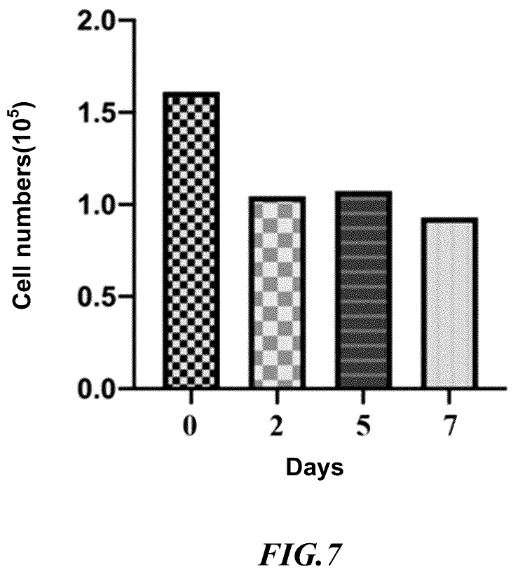

Please refer to FIG. 7, which shows a statistical graph of the survival rate of HUVECs in the vascular bone organoid subjected to compressive stress. As illustrated in FIG. 7, after 2 days of compressive force stimulation using the compression-perfusion fabricator system, the number of HUVECs slightly decreases due to external environmental stress. However, with continued compressive force stimulation for 5 and 7 days, there is no significant decrease in cell survival rate. This result indicates that the HUVECs in the vascular bone organoid subjected to compressive stress can still maintain a good survival rate. Furthermore, the experimental process demonstrates that the compression-perfusion fabricator system described in the present invention can indeed serve as an effective experimental research system.

2.3 The function of HUVECs

### Materials and Methods

In this embodiment, the vascular bone organoid was cultured using the compression-perfusion fabricator system for 2 days. Immunofluorescence (IF) staining using a CD31 (green) antibody was performed to label the HUVECs, combined with DAPI (blue) nuclear staining. CD31 represents the functional marker of HUVECs.

### Results

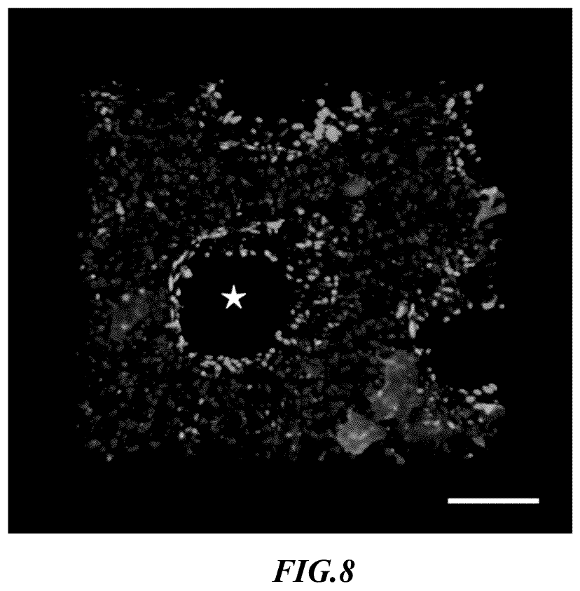

Please refer to FIG. 8, which is the confocal microscope analysis of HUVACs on the vascular bone organoid. The micrograph shows that HUVACs express functional markers (CD31, green) in the vascular bone organoid. The result indicated that HUVACs can grow normally and maintain their function in the vascular bone organoid even if culturing through the compression-perfusion fabricator system.

### EXAMPLE 3 OPCs of the Vascular Bone Organoid

In this example, osteoprogenitor cells (OPCs) were seeded onto a polycaprolactone (PCL) osteogenic mesh scaffold using the process described in Example 1.1. Confocal microscope analysis was performed to observe the growth and differentiation of OPCs.

### Materials and Methods

In this embodiment, OPCs were seeded onto a polycaprolactone (PCL) osteogenic mesh scaffold and cultured for 21 days. Immunofluorescence (IF) staining with an actin antibody was used to label the cytoskeleton of osteoblasts within the PCL osteogenic mesh scaffold, combined with DAPI nuclear staining. The specific immunofluorescence staining protocol follows the steps described in Example 2.1. The primary antibody used was an HRP-conjugated beta-actin mouse monoclonal antibody, diluted at a ratio of 1:2500.

### Results

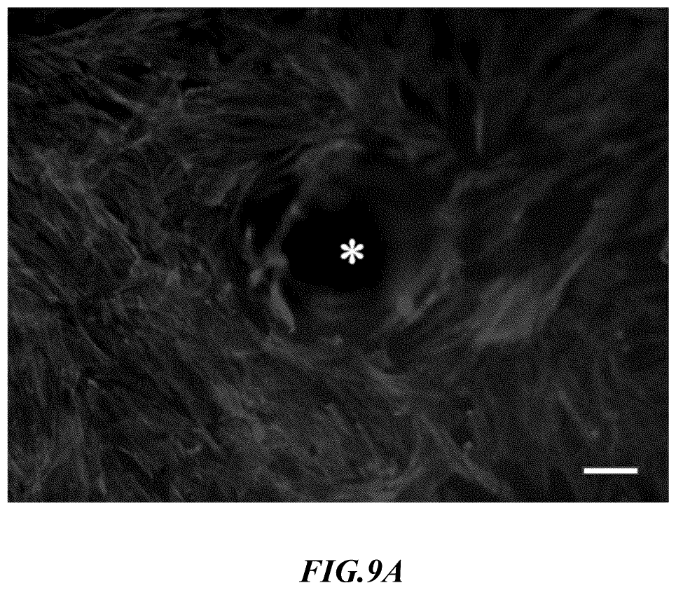

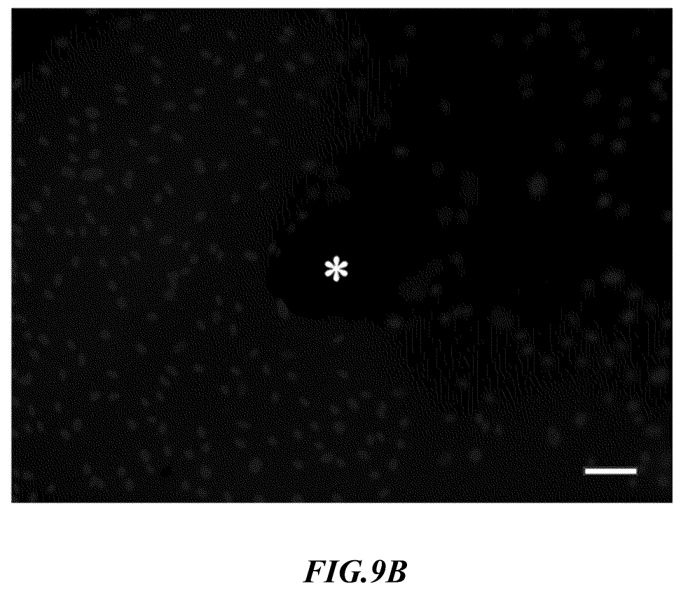

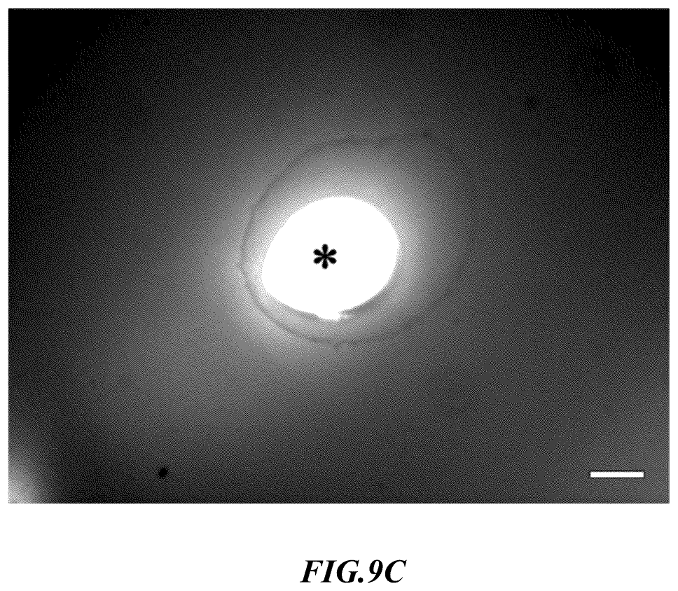

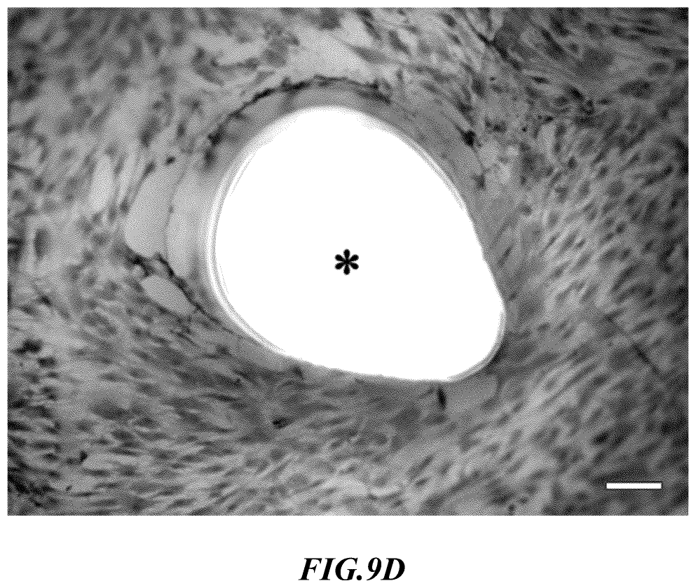

Please refer to FIGS. 9A, 9B, 9C, and 9D, which are micrographs of osteoblasts growing on the surface of the polycaprolactone (PCL) osteogenic mesh scaffold. The asterisks (*) in the images indicate the pore locations of the mesh scaffold. The scale bar in the figures represents 30 μm. In FIG. 9A, the Osteoblast was labeled with a cell tracker (red staining) which binds to the cytoskeleton (actin) before being added to the scaffold. FIG. 9B shows the DAPI nuclear staining (blue). FIG. 9C shows the pore of the PCL osteogenic mesh scaffold observed under a phase contrast microscope, and FIG. 9D shows the expression of alkaline phosphatase in osteoblasts on the PCL osteogenic mesh scaffold observed under an optical microscope. As shown in FIGS. 9A, 9B, 9C, and 9D, osteoprogenitor cells (OPCs) can survive well and normally differentiate into functional osteoblasts within the polycaprolactone (PCL) osteogenic mesh scaffold described in the present invention.

### EXAMPLE 4 Application of the Compression-Perfusion Fabricator System

In this example, the effectiveness of the compression-perfusion fabricator system described in the present invention is confirmed by analyzing the expression of PIEZO1 and endomucin (EMCN) in human umbilical vein endothelial cells (HUVECs). Furthermore, the compression-perfusion fabricator system was used to study the phosphorylation mechanism of yes-associated protein (YAP) in HUVECs. This study aims to demonstrate that the compression perfusion culture system described in the present invention can serve as an effective experimental research platform.

### Materials and Methods

In this embodiment, a vascular mesh scaffold containing human umbilical vein endothelial cells (HUVECs) was prepared using the process described in Example 1.2. The vascular mesh scaffold, containing HUVECs, was then cultured in the compression-perfusion fabricator system described in the present invention. Compressive force stimulation was applied for 0 minute (NC), 30 minutes (C30), 60 minutes (C60), and 120 minutes (C120), respectively, to observe the change of expression of PIEZO1, EMCN, and the dephosphorylation of YAP protein in the cells. The specific experimental steps comprise:
Adding ECM medium to the perfusion chamber of the compression perfusion culture system. Adjusting the compression parameters to a strain of 10% and a voltage of 10 V (frequency 0.2 Hz) to provide the compressive stimulation. Place the compression perfusion culture system in an incubator set at 37° C. with 5% CO₂.

After compression cultivation, remove the medium and wash the vascular mesh scaffold with PBS. Then, cut the vascular mesh scaffold into small pieces and homogenize it using a tissue grinder. Adding 200 μl of 2X RIPA buffer (Bio Basic, Canada, Catalog No. RB4475) and 2 μl of protease inhibitor cocktail (FUTURE, Taiwan, Catalog No. F1PICool). Rotating and mixing at 4° C. for 30 minutes, and centrifuge at 13,200 rpm for 30 minutes. The supernatant obtained is the protein sample.

Performing Western blotting analysis. Using anti-PIEZO1 antibody (Proteintech, USA, Cat. No. 28511-1-AP), anti-endomucin antibody (R&D Systems, USA, Cat. No. AF7206), or anti-phospho-YAP (Ser127) antibody (Cell Signaling Technology, USA, Catalog No. #4911) as the primary antibody, diluted 1:1000. Incubate at 4° C. with gentle shaking for one day. Wash away the unbound primary antibody and add the secondary antibody (Goat anti-rabbit HRP, Jackson ImmunoResearch, USA, Catalog No. 111-005-003), diluted 1:2500. Incubate at room temperature with gentle shaking for 90 minutes. After removing the secondary antibody and washing, proceed with signal detection. The signal detection method involves using chemiluminescence to detect the target protein bands. Applying electrochemiluminescence (ECL) substrate evenly over the entire transfer membrane. Placing the membrane in a chemiluminescence imaging system, adjusting the exposure time for image analysis, and using software to quantitatively analyze the protein expression levels.

### Results

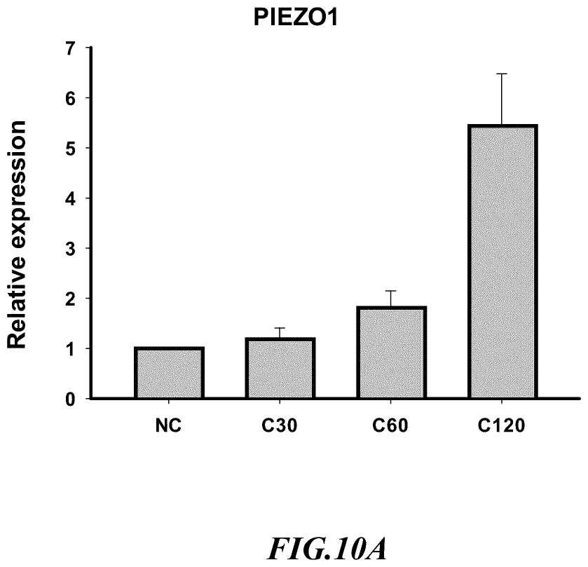

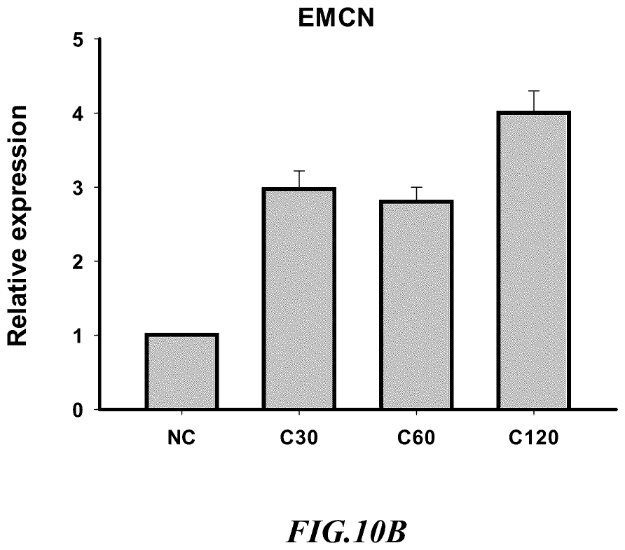

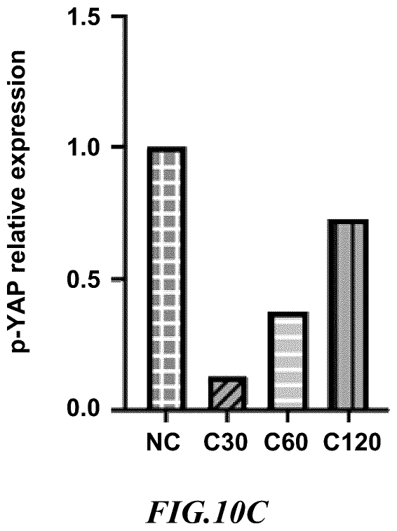

While facing compressive stimulation, HUVECs will express the expression of PIEZO1 and EMCN protein. Please refer to FIGS. 10A and 10B, which show the expression of PIEZO1 and EMCN protein in HUVECs after being subjected to compressive stimulation through the compression-perfusion fabricator system described in the present invention. The expression of PIEZO1 and EMCN protein gradually increases after 30, 60, and 90 minutes of compressive stimulation. The result indicated that the compression-perfusion fabricator system described in the present invention can effectively serve as a platform for compression stimulation.

Please refer to FIG. 10C, which shows the phosphorylation results of YAP protein in HUVECs after being subjected to compressive stimulation using the compression-perfusion fabricator system described in the present invention. The YAP protein in HUVECs undergoes dephosphorylation after 30 minutes of compressive stimulation. Over time, the YAP protein gradually returns to its phosphorylated state (60 minutes and 120 minutes). These experimental designs and methods demonstrate that the compression-perfusion fabricator system described in the present invention can serve as a practical and effective experimental research platform.

### EXAMPLE 5 Mono-Culture and Co-Culture of the Vascular Bone Organoid

In this example, HUVECs were cultured with or without OPCs to verify the influence of cell co-culture (HUVECs and OPCs) in the vascular bone organoid described in the present invention.

### Materials and Methods

In this embodiment, the term “mono-culture” represents a vascular mesh scaffold cultured without osteogenic mesh scaffold, wherein “mono-culture” can be also regarded as using the GelMa containing HUVEC inserted into two PCL plate (without OPCs) for an experiment. In this embodiment, the term “co-culture” represents a vascular mesh scaffold cultured with osteogenic mesh scaffold, wherein “co-culture” can be also regarded as using the vascular bone organoid described in the present invention for an experiment.

In this embodiment, the vascular mesh scaffold containing HUVECs was prepared using the process described in Example 1.2. The vascular bone organoid was prepared using the process described in Example 1.3. All of the samples were cultured in the compression-perfusion fabricator system described in the present invention. Compressive force stimulation was applied for 0 minute (NC), 30 minutes (C30), 60 minutes (C60), and 120 minutes (C120), respectively, to observe the change in the RNA expression level of PIEZO1 and EMCN. RNA of the samples are extracted by Total RNA purification kit (GeneMark, USA, Cat. No. TR01), and the RNA expression level is detected by RT-PCR.

### Results

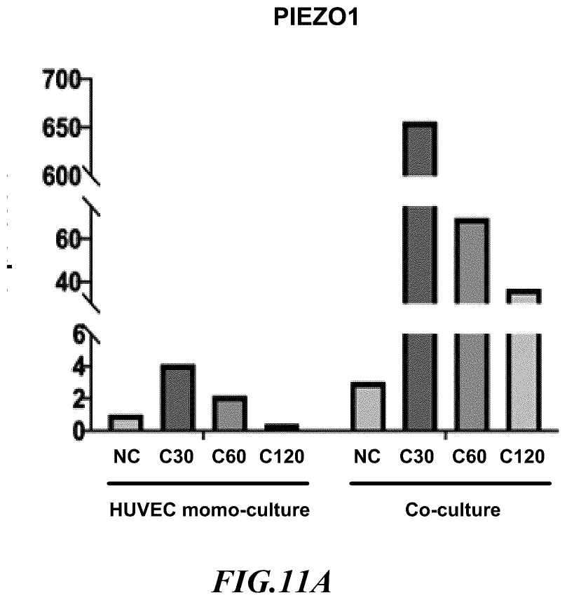

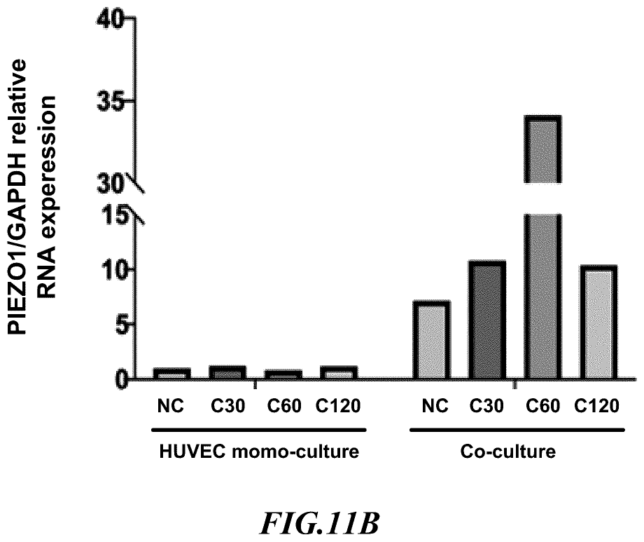

Please refer to FIGS. 11A and 11B. The RNA expression of PIEZO1 and EMCN were increased after compressive stimulation in mono-culture and co-culture indicating that the compression-perfusion fabricator system described in the present invention can serve as an effective experimental research platform. Moreover, the co-culture sample shows a more obvious expression signal than mono-culture indicating that the vascular bone organoid described in the present invention has a more significant response to compressive stimulation which is more similar to the real situation in vivo. The result demonstrates that the vascular bone organoid described in the present invention can serve as a more effective experimental research model.

After culture and experiments, it has been confirmed that the vascular bone organoid described in the present invention supports the survival of osteoprogenitor cells (OPCs) seeded on the first and second osteogenic mesh scaffolds, as well as human umbilical vein endothelial cells (HUVECs) seeded on the vascular mesh scaffold. These cells exhibit normal differentiation and functional responses to external stimuli. Furthermore, the compression-perfusion fabricator system described in the present invention has been experimentally validated to provide mechanical and fluid stress stimulation, simulating the real tissue environment in vivo. Cells cultured using the compression-perfusion fabricator system maintain high survival rates and normal cellular functions, demonstrating that the system can serve as an effective platform for drug development and related research. It has extensive application potential in the field of cell culture.

In summary, the present invention utilizes 3D bioprinting technology to culture human osteoprogenitor cells (OPCs) on a hollow cubic mesh polycaprolactone (PCL) osteogenic mesh scaffold. Additionally, a mixture of human umbilical vein endothelial cells (HUVECs) and gelatin methacryloyl (GelMA) hydrogel is combined with the aforementioned PCL osteogenic mesh scaffold, successfully creating vascular bone organoid capable of normal growth and functionality. Furthermore, the invention provides a compression-perfusion fabricator system that integrates mechanical compression and fluid flow stimulation into cell scaffolds. This system effectively simulates real physiological dynamic environments, enhancing cell vitality, promoting angiogenesis, and reinforcing extracellular matrix mineralization. Consequently, it increases the strength and success rate of artificial organ fabrication. This system can serve as an effective experimental animal replacement research platform or personalized drug screening platform. Moreover, it may contribute to the future development of personalized autologous organ transplantation.

Many changes and modifications in the above described embodiment of the invention can, of course, be carried out without departing from the scope thereof. Accordingly, to promote the progress in science and the useful arts, the invention is disclosed and is intended to be limited only by the scope of the appended claims.

What is claimed is:

1. A compression-perfusion fabricator system, comprising:
a compression device comprising a cell growth area, a first electric gripper jaw, a second electric gripper jaw, and a micromotor, wherein the cell growth area is positioned between the first and second electric gripper jaws and the micromotor is electrically connected to the first and second electric gripper jaws;
a perfusion chamber for loading cell culture medium, wherein the cell growth area and the first and second electric gripper jaws are arranged in the perfusion chamber;
a perfusion device comprising a culture medium bottle, a culture medium recycling bottle, and a peristaltic pump, wherein the peristaltic pump is connected to the perfusion chamber, the culture medium bottle, and the culture medium recycling bottle through tubes; and
a microcomputer driver comprising a control panel, wherein the microcomputer driver is electrically connected to the peristaltic pump and the micromotor;
wherein the compression device and the perfusion device are controlled and adjusted through the microcomputer driver to simulate the dynamic microenvironment in the human body.

2. The system of claim 1, wherein the system further comprises at least one support bracket for fixing the compression device, the perfusion chamber, and the perfusion device.

3. The system of claim 1, wherein the microcomputer driver further comprises a flow sensor.

4. The system of claim 1, wherein the system is used for culturing and differentiating at least one stem cell, wherein the stem cell is selected from the group consisting of osteoprogenitor cells (OPCs), human umbilical vein endothelial cells (HUVEC), mesenchymal stem cells (MSC), and hematopoietic stem cells.

5. The system of claim 1, wherein the perfusion device applies pressure through the peristaltic pump to transport fresh culture medium from the culture medium bottle into the perfusion chamber via tubing, and to transport the culture medium from the perfusion chamber into the culture medium recycling bottle via tubing.

6. The system of claim 1, wherein the microcomputer driver controlled the peristaltic pump by adjusting control parameters displayed on the control panel.

7. The system of claim 6, wherein the control parameters comprise liquid flow rate, liquid flow volume, and perfusion time.

8. The system of claim 1, wherein the compression device shortens the distance between the first electric gripper jaw and the second electric gripper jaw through the micromotor to generate pressing force on the cell growth area.

9. The system of claim 1, wherein the microcomputer driver controlled the micromotor by adjusting control parameters displayed on the control panel.

10. The system of claim 9, wherein the control parameters comprise the position of the first electric gripper jaw, the position of the second electric gripper jaw, the compression time of the first and second electric gripper jaws, the compression rate of the first and second electric gripper jaws, the amount of the compressive force, the duration of the compressive force, the frequency of the compressive force, and the amount of deformation.

11. The system of claim 1, wherein the compression device secures an organoid in the cell growth area with the first and second electric gripper jaws and immerses the organoid in the perfusion chamber.

12. The system of claim 11, wherein the organoid is a vascular bone organoid, comprising:
a first osteogenic mesh scaffold seeded with osteoprogenitor cells (OPCs);
a second osteogenic mesh scaffold seeded with osteoprogenitor cells (OPCs); and
a vascular mesh scaffold seeded with human umbilical vein endothelial cells (HUVEC) and placed between the first and second osteogenic mesh scaffolds.

13. The system of claim 1, wherein the system is used for culturing an organoid.

14. A vascular bone organoid, comprising:
a first osteogenic mesh scaffold made of a first biocompatible material, wherein the first osteogenic mesh scaffold is seeded with osteoprogenitor cells (OPCs);
a second osteogenic mesh scaffold made of a second biocompatible material, wherein the second osteogenic mesh scaffold is seeded with osteoprogenitor cells (OPCs); and
a vascular mesh scaffold placed between the first and second osteogenic mesh scaffolds, wherein the vascular mesh scaffold is made of a third biocompatible material and seeded with human umbilical vein endothelial cells (HUVEC).

15. The vascular bone organoid of claim 14, wherein the first osteogenic mesh scaffold, the second osteogenic mesh scaffold, and the vascular mesh scaffold are made by 3D printing.

16. The vascular bone organoid of claim 14, wherein the first, second, and third biocompatible materials are independently selected from the group consisting of polycaprolactone, gelatin methacryloyl (GelMa), Pluronic F127, chitosan, collagen, and alginate.

17. The vascular bone organoid of claim 14, wherein the vascular bone organoid is cultured through the compression-perfusion fabricator system as described in claim 1, wherein the vascular bone organoid is cultured in the cell growth area.

18. A method for culturing organoids, comprising:
providing a compression-perfusion fabricator system as described in claim 1;
culturing at least one stem cell on the cell growth area; and
utilizing the peristaltic pump for fluid perfusion and employing the micromotor for compressive stimulation, to simulate the dynamic microenvironment of cell culture in the human body.

19. The method of claim 18, wherein the stem cell is selected from the group consisting of osteoprogenitor cells (OPCs), human umbilical vein endothelial cells (HUVECs), mesenchymal stem cells (MSCs), and hematopoietic stem cells.

20. The method of claim 18, wherein the method is used for culturing the vascular bone organoid as described in claim 8, wherein the vascular bone organoid is cultured in the cell growth area.
